SEQUENCE LISTING (2 sequences)

SEQ ID NO: 1 - DNA, 609 bp, Ustilago maydis
  CDS (1)..(609)
atgccgtcgtccaactgggcgtcgacgcctccgattccaagcgacaaacgtccgatcgtgctgtcgggtccttcgggtgtgggcaagagtacacttctgaaaaagttgtttcaagagttccccaatgactttggtttttccgtatctgatacaacaagagaccctcgaccaggcgaggtccgcggccagtcgtaccactacgtctcacagcaagaattccaagaccttgttcaacagggcgcgtttctcgaacacgccaagtttggcggcaaccgatacggcactaccgccaaggcggtggctgacgtatccaccgaaggccgtcgcgctatcctcgacatcgacgctcaaggcgtcaagctcatcaaggccaaccacgcttcgttgaacccgatctacatctttatctctccaccttcgttttcgactctcaagcagaggttgataggtcgaggcaccgagacaccagagtcagtcaacaagaggctctctatggctgccaccgagatggcttatgccagggaaaagggagcgcacgattgggtcattgtcaatgacgatttggaaaaggcttatggcttgttgaagaaggcgatcaacgaaactttg

SEQ ID NO: 2 - protein, 203 aa, Ustilago maydis
MPSSNWASTPPIPSDKRPIVLSGPSGVGKSTLLKKLFQEFPNDFGFSVSDTTRDPRPGEVRGQSYHYVSQQEFQDLVQQGAFLEHAKFGGNRYGTTAKAVADVSTEGRRAILDIDAQGVKLIKANHASLNPIYIFISPPSFSTLKQRLIGRGTETPESVNKRLSMAATEMAYAREKGAHDWVIVNDDLEKAYGLLKKAINETL

(19) United States
(12) Patent Application Publication
(10) Pub. No.: US 2005/0019850 A1
(43) Pub. Date: Jan. 27, 2005

(54) Method for identifying fungicidally active compounds based on guanylate kinases

(76) Inventors: Martin Vaupel, Leichlingen (DE); Edda Koopmann, Langenfeld (DE); Verena Vollenbroich, Mulheim (DE)

Correspondence Address: BAYER MATERIAL SCIENCE LLC, 100 BAYER ROAD, PITTSBURGH, PA, 15205, US

(21) Appl. No.: 10/881,046

(22) Filed: Jun. 30, 2004

(30) Foreign Application Priority Data
Jul. 4, 2003 (DE) 10330233.6

(51) Int. Cl. C12Q 1/48; A01N 57/00

(52) U.S. Cl. 435/15; 514/114

(57) ABSTRACT

The invention relates to a method for identifying fungicides, to the use of fungal guanylate kinase for identifying fungicides, and to the use of guanylate kinase inhibitors as fungicides.

The invention relates to a method for identifying fungicides, to the use of fungal guanylate kinase for identifying fungicides, and to the use of guanylate kinase inhibitors as fungicides.

Undesired fungal growth which leads every year to considerable damage in agriculture can be controlled by the use of fungicides. The demands made on fungicides have increased constantly with regard to their activity, costs and, above all, ecological soundness. There exists therefore a demand for new substances or classes of substances which can be developed into potent and ecologically sound new fungicides. In general, it is customary to search for such new lead structures in greenhouse tests. However, such tests require a high input of labour and a high financial input. The number of the substances which can be tested in the greenhouse is, accordingly, limited. An alternative to such tests is the use of what are known as high-throughput screening (HTS) methods. This involves testing a large number of individual substances with regard to their effect on cells, individual gene products or genes in an automated method. When certain substances are found to have an effect, they can be studied in conventional screening methods and, if appropriate, developed further.

Advantageous targets for fungicides are frequently searched for in essential biosynthetic pathways. Ideal fungicides are, moreover, those substances which inhibit gene products which have a decisive importance in the manifestation of the pathogenicity of a fungus.

It was therefore an aim of the present invention to identify, and make available, a suitable new target for potential fungicidal active compounds and to provide a method, based thereon, which makes possible the identification of modulators of this target which can eventually be used as fungicides.

Guanylate kinase (E.C. 2.7.4.8), also known as deoxyguanylate kinase, 5′-GMP kinase, GMP kinase, guanosinmonophosphate kinase, ATP:GMP phosphotransferase, catalyzes the reaction of ATP and GMP to ADP and GDP. The reaction of guanylate kinase is an essential stage in the recycling of GMP and in the production of GTP (Li et al., 1996).

Genes for guanylate kinase were cloned from a variety of organisms, inter alia from various fungi, for example from Saccharomyces cerevisiae (Swissprot Accession No.: P15454), Neurospora crassa (Genbank Accession No. AABX01000205), Schizosaccharomyces pombe (Swissprot Accession No.: Q9P615), mice (Swissprot Accession No.: Q64520) or Homo sapiens (Swissprot Accession No.: Q16774). The sequence similarities are significant within the eukaryotic classes.

Guanylate kinase has already been isolated, expressed, purified, characterized and crystallized, for example from yeast (Moriguchi et al., 1981; Berger et al., 1989; Stehle et al., 1990; Konrad, 1992; Blaszczyk et al., 2001).

It was an object of the present invention to identify novel targets of fungicides in fungi, in particular in phytopathogenic fungi, and to make available a method in which inhibitors of such a target, or polypeptide, can be identified and assayed for their fungicidal characteristics. This object was achieved by isolating, from a phytopathogenic fungus, the nucleic acid encoding guanylate kinase, obtaining the polypeptide encoded by it and providing a method by means of which inhibitors of this enzyme can be identified. The inhibitors identified in this method can be employed in vivo against fungi.

### DESCRIPTION OF THE FIGURES

FIG. 1: Homology between guanylate kinases from a variety of fungi. Frames represent regions with an accurately matching sequence (consensus sequence). In the Ustilago maydis sequence, the amino acids corresponding to the Prosite motif for guanylate kinases were also identified. S_{—}pombe: Saccharomyces pombe. N_{—}crassa: Neurospora crassa. Yeast: Saccharomyces cerevisiae. Ustilago: Ustilago maydis.

FIG. 2: Heterologous expression of the guanylate kinase in E. coli BL21(DE3)pLysS. The overexpressed MBP fusion protein is approximately 64 kDa in size. A size marker was applied in Lane 1. Lane 2: membrane fraction 4 hours after induction with IPTG, cell disruption and separation of membrane and cytoplasm fraction by centrifugation; Lane 3: cytoplasm fraction of the overexpressed guanylate kinase 4 hours after induction with IPTG, cell disruption and separation of membrane and cytoplasm fraction by means of centrifugation; Lane 4: cytoplasm fraction of the overexpressed guanylate kinase 4 hours after induction with IPTG, cell disruption and separation of membrane and cytoplasm fraction by means of centrifugation, but diluted 1:1 with buffer; Lane 5: eluate when the cytoplasm fraction is applied to the amylose resin; Lane 6: wash fractions after application of the cytoplasm fraction to the amylose resin; Lanes 7-10: elution fractions with purified guanylate kinase.

FIG. 3: Determination of the K_{M} value for GMP (A) and ATP (B). Lineweaver/Burk representation of the data: 1/Vₒ=1/Vₘₐₓ+1/S×(K_{M}/Vₘₐₓ), wherein Vₒ is the initial reaction rate, Vₘₐₓ the maximum conversion rate possible and S the substrate concentration. Vₘₐₓ and K_{M} can then be read as the intercepts on the horizontal and the vertical axes 1/Vₘₐₓ and 1/K_{M}, respectively. The K_{M} value for GMP is 0.04 mM, for ATP 0.1 mM.

SEQ ID No. 1: nucleic acid sequence encoding the Ustilago maydis guanylate kinase.

SEQ ID No. 2: amino acid sequence of the Ustilago maydis guanylate kinase.

Definitions

The term “homology” or “identity” is understood as meaning the number of matching amino acids (identity) with other proteins, expressed in per cent. Preferably, the identity is determined by comparing a given sequence with other proteins with the aid of computer programs. If sequences which have been compared with each other differ in length, the identity is to be determined in such a way that the number of amino acids which the shorter sequence shares with the longer sequence determines the percentage of identity. The identity can be determined routinely by known computer programs which are available to the public, such as, for example, ClustalW (Thompson et al., Nucleic Acids Research 22 (1994), 4673-4680). ClustalW is made available to the public by Julie Thompson (Thompson@EMBL-Heidelberg.DE) and Toby Gibson (Gibson@(2EMBL-Heidelberg.DE), European Molecular Biology Laboratory, Meyerhofstrasse 1, D 69117 Heidelberg, Germany. ClustalW can also be downloaded from various internet pages, inter alia the IGBMC (Institut de Génétique et de Biologie Moléculaire et Cellulaire, B.P.163, 67404, Illkirch Cedex, France; ftp://ftp-igbmc.u-strasbg/fr/pub/) and the EBI (ftp://ftp.ebi.ac.uk/pub/software/) and all mirrored internet pages of the EBI (European Bioinformatics Institute, Wellcome Trust Genome Campus, Hinxton, Cambridge CB10 1SD, UK). If the ClustalW computer program of version 1.8 is used to determine the identity between, for example, a given reference protein and other proteins, the following parameters are to be set: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP. A possibility of finding similar sequences is to carry out sequence database searches. Here, one or more sequences is/are set as what is known as a query. This query sequence is then compared with sequences which are present in the selected databases, using statistics computer programs. Such blast searches are known to the skilled worker and can be carried out at various providers. If such a blast search is carried out for example at the NCBI (National Center for Biotechnology Information, http://www.ncbi.nlm.nih.gov/), the standard settings which are set for each comparison query should be used. For protein sequence comparisons (“blastp”), these settings are: Limit entrez=not activatet; Filter=low complexity activated; Expect value=10; word size=3; Matrix=BLOSUM62; Gap costs: Existence=11, Extension=1. The result of such a query shows not only other parameters, but also the amount of identity between the query sequence and the similar sequences represented in the databases. A protein according to the invention is therefore understood as meaning, in connection with the present invention, those proteins which, when using at least one of the above-described methods for determining identity, have at least 70%, preferably at least 75%, especially preferably at least 80%, further preferably at least 85% and in particular at least 90% identity.

The term “complete guanylate kinase” as used in the present context describes a guanylate kinase which is encoded by a complete encoding region of the transcription unit comprising an ATG start codon and comprising all information-bearing exon regions of the gene present in the source organism and encoding a guanylate kinase, and the signals required for correct transcriptional termination.

The term “biological activity of a guanylate kinase” as used in the present context refers to the ability of a polypeptide to catalyse the above-described reaction, i.e. the conversion of ATP and GMP to ADP and GDP (ATP+GMP [symbol] ADP+GDP). A GMP binding motif is characteristic of guanylate kinases.

The term “active fragment” as used in the present context describes nucleic acids encoding guanylate kinase which are no longer complete, but still encode polypeptides with the biological activity of a guanylate kinase and which are capable of catalysing a reaction characteristic of guanylate kinase, as described above. Such fragments are shorter than the above-described complete nucleic acids encoding guanylate kinase. In this context, nucleic acids may have been removed both at the 3′ and/or 5′ ends of the sequence, or else parts of the sequence which do not have a decisive adverse effect on the biological activity of guanylate kinase may have been deleted, i.e. removed. A lower or else, if appropriate, an increased activity which still allows the characterization or use of the resulting guanylate kinase fragment is considered as sufficient for the purposes of the term as used herein. The term “active fragment” may likewise refer to the amino acid sequence of guanylate kinase and in this case, it applies analogously to what has been said above for those polypeptides which no longer contain certain portions in comparison with the above-described complete sequence, but where no decisive adverse effect is exerted on the biological activity of the enzyme. The fragments may differ with regard to their length.

The term “guanylase kinase inhibition assay” or “inhibition assay” as used in the present context refers to a method or a test which allows the identification of the inhibition of the enzymatic activity of a polypeptide with the activity of a guanylate kinase by one or more chemical compounds (candidate compound(s)), whereby the chemical compound can be identified as a guanylate kinase inhibitor.

The term “gene” as used in the present context is the name for a segment from the genome of a cell which is responsible for the synthesis of a polypeptide chain.

The term “fungicide” or “fungicidal” as used in the present context refers to chemical compounds which are suitable for controlling fungi, in particular phytopathogenic fungi. Such phytopathogenic fungi are mentioned hereinbelow, the enumeration not being final:

Plasmodiophoromycetes, Oomycetes, Chytridiomycetes, Zygomycetes, Ascomycetes, Basidiomycetes and Deuteromycetes, for example

Pythium species such as, for example, Pythium ultimum, Phytophthora species such as, for example, Phytophthora infestans, Pseudoperonospora species such as, for example, Pseudoperonospora humuli or Pseudoperonospora cubensis, Plasmopara species such as, for example, Plasmopara viticola, Bremia species such as, for example, Bremia lactucae, Peronospora species such as, for example, Peronospora pisi or P. brassicae, Erysiphe species such as, for example, Erysiphe graminis, Sphaerotheca species such as, for example, Sphaerotheca fuliginea, Podosphaera species such as, for example, Podosphaera leucotricha, Venturia species such as, for example, Venturia inaequalis, Pyrenophora species such as, for example, Pyrenophora teres or P. graminea (conidial form: Drechslera, syn: Helminthosporium), Cochliobolus species such as, for example, Cochliobolus sativus (conidial form: Drechslera, syn: Helminthosporium), Uromyces species such as, for example, Uromyces appendiculatus, Puccinia species such as, for example, Puccinia recondita, Sclerotinia species such as, for example, Sclerotinia sclerotiorum, Tilletia species such as, for example, Tilletia caries, Ustilago species such as, for example, Ustilago nuda or Ustilago avenae, Pellicularia species such as, for example, Pellicularia sasakii, Pyricularia species such as, for example, Pyricularia oryzae, Fusarium species such as, for example, Fusarium culmorum, Botrytis species, Septoria species such as, for example, Septoria nodorum, Leptosphaeria species such as, for example, Leptosphaeria nodorum, Cercospora species such as, for example, Cercospora canescens, Alternaria species such as, for example, Alternaria brassicae or Pseudocercosporella species such as, for example, Pseudocercosporella herpotrichoides.

Others which are of particular interest are, for example, Magnaporthe grisea, Cochliobulus heterostrophus, Nectria hematococcus and Phytophthora species.

However, fungicidal active substances which are found with the aid of the guanylate kinase according to the invention, can also interact with guanylate kinase from fungal species which are pathogenic for humans, it not being necessary for the interaction with the different guanylate kinases which occur in these fungi to be always equally pronounced.

The present invention therefore also relates to the use of guanylate kinase inhibitors for the preparation of compositions for the treatment of diseases caused by fungi which are pathogenic for humans.

Of particular interest in this context are the following fungi which are pathogenic to humans and which may cause the symptoms stated hereinbelow: Dermatophytes such as, for example, Trichophyton spec., Microsporum spec., Epidermophyton floccosum or Keratomyces ajelloi, which cause, for example, Athlete'foot (Tinea pedis), yeasts such as, for example, Candida albicans, which causes soor oesophagitis and dermatitis, Candida glabrata, Candida krusei or Ciyptococcus neoformans, which may cause, for example, pulmonal cryptococcosis or else torulosis, moulds such as, for example, Aspergillus fumigatus, A. flavus, A. niger, which cause, for example, bronchopulmonary aspergillosis or fungal sepsis, Mucor spec., Absidia spec., or Rhizopus spec., which cause, for example, zygomycoses (intravasal mycoses), Rhinosporidium seeberi, which causes, for example, chronic granulomatous pharyngitis and tracheitis, Madurella mycetomatis, which causes, for example, subcutaneous mycetomas, Histoplasma capsulatum, which causes, for example, reticuloendothelial cytomycosis and Darling's disease, Coccidioides immitis, which causes, for example, pulmonary coccidioidomycosis and sepsis, Paracoccidioides brasiliensis, which causes, for example, South American blastomycosis, Blastomyces dermatitidis, which causes, for example, Gilchrist's disease and North American blastomycosis, Loboa loboi, which causes, for example, keloid blastomycosis and Lobo's disease, and Sporothrix schenckii, which causes, for example, sporotrichosis (granulomatous dermal mycosis).

Fungicidal active substances which can be found with the aid of a guanylate kinase obtained from a specific fungus, in the present case from Ustilago maydis, can therefore also interact with guanylate kinase from a large number of other fungal species, in particular also with phytopathogenic fungi, it not always being necessary for the interaction with the different guanylate kinases which occur in these fungi to be equally pronounced. This explains, inter alia, the selectivity which has been observed in the substances which act on this enzyme.

The term “homologous promoter” as used in the present context refers to a promoter which controls the expression of the respective gene in the starting organism. The term “heterologous promoter” as used in the present context refers to a promoter which has properties other than the promoter which controls the expression of the relevant gene in the starting organism.

The term “competitor” as used in the present context refers to the property of the compounds to compete with other, possibly yet to be identified, compounds for binding to guanylate kinase and to displace the latter, or to be displaced by the latter, from the enzyme.

The term “modulator” as used in the present context is the generic term for inhibitors or activators. Modulators can be small organochemical molecules, peptides or antibodies which bind to the polypeptides according to the invention or influence their activity. Moreover, modulators can be small organochemical molecules, peptides or antibodies which bind to a molecule which, in turn, binds to the polypeptides according to the invention, thus influencing their biological activity. Modulators can be natural substrates and ligands, or structural or functional mimetics of these. Preferably, the term “modulator” as used in the present context however refers to those molecules which do not constitute the natural substrates or ligands.

### DESCRIPTION OF THE INVENTION

In the present invention, the complete sequence of a guanylate kinase from the phytopathogenic fungus Ustilago maydis is made available for the first time, which makes possible the more detailed study of guanylate kinases, in particular from phytopathogenic fungi, and thus the detection of a novel target protein for the identification of novel fungicidal active compounds.

Despite extensive research into guanylate kinase, it was previously unknown that guanylate kinase may be a target protein for fungicidally active substances in fungi. The present invention thus shows for the first time that guanylate kinase constitutes an enzyme which is important in particular for fungi and which is therefore particularly suitable as target protein for the search for further, and improved, fungicidally active compounds.

Inhibitors of guanylate kinase with a fungicidal activity have not been described to date. While various publications point out the particular role which guanylate kinase plays in the metabolism of organisms, such as, for example, the yeast Saccharomyces cerevisiae and describe that the destruction of the yeast gene which encodes guanylate kinase is lethal for S. cerevisiae, none of the publications discusses the question whether the enzyme guanylate kinase can be influenced, for example inhibited, by active compounds and whether a treatment of fungi in vivo with an active compound which modulates guanylate kinase is possible in order to control fungi. Thus, guanylate kinase has not been described as yet as target protein for fungicides. No active compounds which have fungicidal activity and which target guanylate kinase are known.

It has been demonstrated within the scope of the present invention with the aid of knock-out experiments (Example 1) that guanylate kinase can be, in phytophatogenic fungi, a target for fungicidal active compounds, that inhibition of guanylate kinase might lead to damage or destruction of the fungus. In further experiments which focused on the sensitivity of guanylate kinase to active compounds in vitro and also in vivo, it was furthermore possible to identify the enzyme guanylate kinase as a polypeptide which can be used for identifying modulators or inhibitors of its enzymatic activity in suitable test methods, which is not naturally the case in a variety of targets which are theoretically of interest.

This is why, within the scope of the present invention, a method has been developed which is suitable for determining the activity of guanylate kinase and the inhibition of this activity in what is known as an inhibition assay, thus identifying inhibitors of the enzyme, for example in HTS and UHTS methods, and testing the fungicidal properties. It has also been shown within the scope of the present invention that the inhibitors of guanylate kinase from fungi can be used as fungicides.

It has furthermore been found within the scope of the present invention that guanylate kinase can also be inhibited in vivo by active compounds, and that a fungal organism which is treated with these active compounds can be damaged or destroyed by the treatment with these active compounds. The inhibitors of a fungal guanylate kinase can thus be used as fungicides in crop protection, or else as antimycotics in pharmaceutical indications. For example, it is demonstrated in the present invention that inhibition of guanylate kinase with one of the substances identified in a method according to the invention leads to the death of the treated fungi in synthetic media or on the plant.

The inhibitors of a fungal guanylate kinase can be used as fungicides, in particular in crop protection, or else as antimycotics in pharmaceutical indications. For example, it is demonstrated in the present invention that inhibition of guanylate kinase with a substance identified in a method according to the invention leads to the death of the treated fungi in synthetic media or on the plant.

Guanylate kinase can be obtained from various phytopathogenic fungi or else fungi which are pathogenic to humans or animals, for example from fungi such as the phytopathogenic fungus U. maydis. To prepare guanylate kinase from fungi, the gene can be expressed for example recombinantly in Escherichia coli, and an enzyme preparation can be prepared from E. coli cells (Example 2). Preferably, guanylate kinases from phytopathogenic fungi are used for identifying fungicides which can be employed in crop protection. If it is intended to identify fungicides or antimycotics to be used in pharmacological indications, it is recommended to employ guanylate kinases from fungi which are pathogenic to humans or animals.

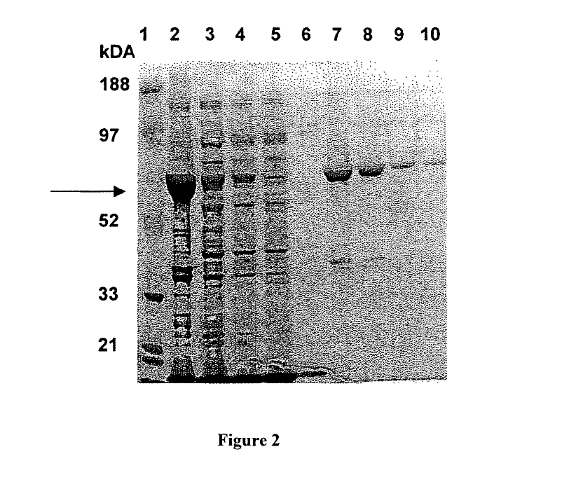

To express the polypeptide GUK1, which is encoded by guk1, the corresponding ORF was thus amplified from total RNA by RT-PCR using methods known to the skilled worker and selected primers. The DNA in question was cloned into the vector pDESTMC2 (Gateway Vector from Invitrogen, provided with an N-terminal MBP tag (=maltose binding protein tag)). The resulting plasmid pGuk1 contains the complete coding sequence of guk1 in n-terminal fusion with the MBP tag from the vector. The GUK1 fusion protein has a calculated mass of approximately 64 kDa (Example 2 and FIG. 2).

The plasmid pGukl was then used for recombinantly expressing GUK1 in E. coli BL21 (DE3)pLysS (Example 2).

The present invention thus also provides a further complete genomic sequence of a phytopathogenic fungus encoding a guanylate kinase, and describes its use, or the use of the polypeptide encoded thereby, for identifying inhibitors of the enzyme.

The present invention therefore also relates to a polypeptide with the enzymatic function of a nucleic acid from the fungus Ustilago maydis which encodes a guanylate kinase.

Guanylate kinases can be divided into homologous regions. Typically of guanylate kinases is a highly conserved region containing two arginines and one tyrosine, both of which are involved in binding GMP.

This GMP binding site is a sequence characteristic which is characteristic of guanylate kinases. Such a motif was identified by a suitable search in the PROSITE database (Hofmann K., Bucher P., Falquet L., Bairoch A. (1999) “The PROSITE database, its status in 1999”. Nucleic Acids Res. 27, 215). This can be shown as follows:
T-[ST]-R-x(2)-[KR]-x(2)-[DE]-x(2)-G-x(2)-Y-x-[FY]-[LIVMK]
PROSITE allows the assignation of a function to polypeptides and is thus suitable for recognizing guanylate kinases as such.

The Prosite motif is shown using the one-letter code. The symbol “x” represents a position at which any amino acid is accepted. A variable position at which various specific amino acids are accepted is shown in square brackets “[ . . . ]”, the amino acids which are possible at this position being enumerated. Amino acids which are not accepted at a specific position, in contrast, are shown in curly brackets “{ . . . }”. A hyphen “-” separates the individual elements or positions of the motif. If a specific position is repeated, for example “x” several times in succession, this can be shown by showing the number of repetitions within brackets after the x, for example “x (3)”, which represents “x-x-x”.

Thus, a Prosite motif ultimately represents the components of a consensus sequence and distances between the amino acids involved, and is therefore typical of a particular class of enzymes. With reference to this motif, and based on the nucleic acids according to the invention, further polypeptides from phytopathogenic fungi which belong to the same class as the polypeptide according to the invention can be identified or assigned and which can thus also be used in accordance with the invention.

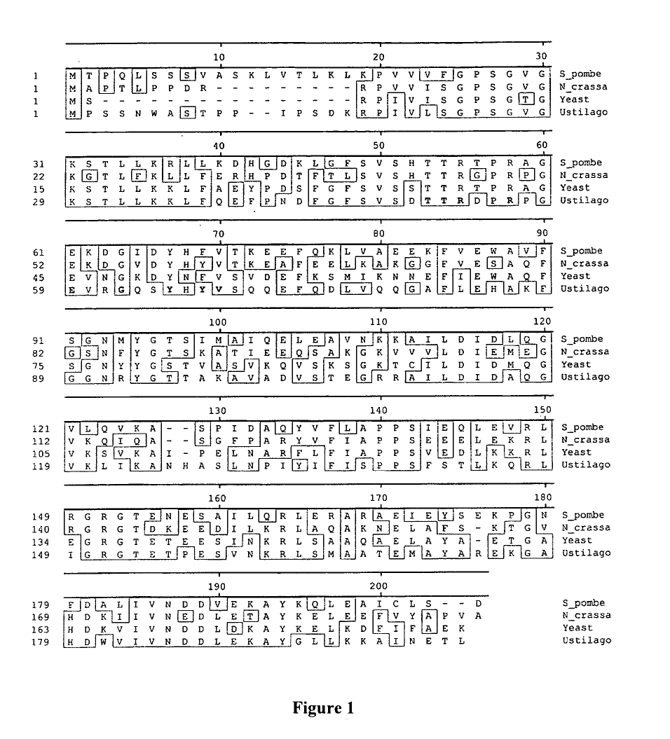

In the case of the U. maydis guanylate kinase, this motif is likewise present in S. cerevisiae, S. pombe or N. crassa (see FIG. 1). The specific consensus sequence for a guanylate kinase according to the invention which can be used for identifying or assigning further polypeptides according to the invention is therefore particularly preferably
T-T-R-x(2)-R-x(2)-E-x(2)-G-x(2)-Y-x-Y-V.

The abovementioned Prosite motif, or the specific consensus sequence, are typical of the polypeptides according to the invention which can be defined in terms of structure with reference to these consensus sequences and can thus also be identified unambiguously.

The present invention therefore also relates to polypeptides from phytopathogenic fungi with the biological activity of a guanylate kinase which encompass the abovementioned Prosite motif T-[ST]-R-x(2)-[KR]-x(2)-[DE]-x(2)-G-x(2)-Y-x-[FY]-[LIVMK], preferably those polypeptides which encompass the abovementioned motif T-T-R-x(2)-R-x(2)-E-x(2)-G-x(2)-Y-x-Y-V.

Owing to the homologies which exist in species-specific nucleic acids encoding guanylate kinases, it is also possible to identify, and use, guanylate kinases from other phytopathogenic fungi in order to achieve the above aim, i.e. they can likewise be used for identifying guanylate kinase inhibitors which, in turn, can be used as fungicides in crop protection. However, it is also feasible to use another fungus which is not phytopathogenic, or its guanylate kinase or the sequence encoding it, in order to identify fungicidally active guanylate kinase inhibitors. Owing to the sequence as shown in SEQ ID No: 1 shown herein, or any primers derived therefrom, and, if appropriate, using the above-shown Prosite motif, it is possible for the skilled worker to obtain and to identify further nucleic acids encoding guanylate kinases from other (phytopathogenic) fungi, for example by means of PCR. Such nucleic acids and their use in methods for identifying fungicidal active compounds are considered as being encompassed by the present invention.

With the aid of the nucleic acid sequence according to the invention and sequences from other phytopathogenic fungi, which sequences are obtained in accordance with the abovementioned methods, it is possible to identify further guanylate-kinase-encoding nucleic acid sequences from other fungi.

The present invention therefore relates to nucleic acids from phytopathogenic fungi which encode a polypeptide with the enzymatic activity of a guanylate kinase, in particular to polypeptides which contain the abovementioned motifs.

Preferably, the present invention relates to nucleic acids from the phytopathogenic fungal species mentioned above under the definitions which encode a polypeptide with the enzymatic activity of a guanylate kinase.

In particular, the present invention relates to the guanylate-kinase-encoding Ustilago maydis nucleic acid with the SEQ ID No: 1 and to the nucleic acids encoding the polypeptides as shown in SEQ ID No: 2 or active fragments thereof.

The nucleic acids according to the invention are, in particular, single- or double-stranded deoxyribonucleic acids (DNAs) or ribonucleic acids (RNAs). Preferred embodiments are fragments of genomic DNA, and cDNAs.

Especially preferably, the nucleic acids according to the invention comprise a sequence from phytopathogenic fungi encoding a polypeptide with the enzymatic activity of a guanylate kinase selected from
a) a sequence as shown in SEQ ID No: 1,
b) sequences encoding a polypeptide which comprises the amino acid sequence shown in SEQ ID No: 2,
c) sequences encoding a polypeptide which comprises the motif T-[ST]-R-x(2)-[KR]-x(2)-[DE]-x(2)-G-x(2)-Y-x-[FY]-[LIVMK] or the motif T-T-R-x(2)-R-x(2)-E-x(2)-G-x(2)-Y-x-Y-V,
d) sequences which hybridize with the sequences defined under a) and b) at a hybridization temperature of 42-65° C., and
e) sequences which have at least 80%, preferably at least 85% and especially preferably at least 90% identity with the sequences defined under a) and b).

As already detailed above, the present invention is not only limited to the Ustilago maydis guanylate kinase. Analogously, and in a manner known to the skilled worker, it is also possible to obtain polypeptides with the activity of a guanylate kinase from other fungi, preferably from phytopathogenic fungi, and such polypeptides can then be employed for example in a method according to the invention. However, it is preferred to use the Ustilago maydis guanylate kinase.

The present invention furthermore relates to DNA constructs comprising a nucleic acid according to the invention and a homologous or heterologous promoter.

The choice of heterologous promoters depends on whether prokaryotic or eukaryotic cells or cell-free systems are used for the expression. Examples of heterologous promoters are the cauliflower mosaic virus 35S promoter for plant cells, the alcohol dehydrogenase promoter for yeast cells, and the T3, T7 or SP6 promoters for prokaryotic cells or cell-free systems.

Preferably, fungal expression systems such as, for example, the Pichia pastoris system should be used, transcription being driven in this case by the methanol-inducible AOX promoter.

The present invention furthermore relates to vectors which contain a nucleic acid according to the invention, a regulatory region according to the invention or a DNA construct according to the invention. All of the phages, plasmids, phagemids, phasmids, cosmids, YACs, BACs, artificial chromosomes or particles which are suitable for particle bombardment which are used in molecular biological laboratories can be used as vectors.

Examples of preferred vectors are the p4XXprom. vector series (Mumberg et al., 1995) for yeast cells, pSPORT vectors (Life Technologies) for bacterial cells, or the Gateway vectors (Life Technologies) for various expression systems in bacterial cells, plants, P. pastoris, S. cerevisiae or insect cells.

The present invention also relates to host cells which contain a nucleic acid according to the invention, a DNA construct according to the invention or a vector according to the invention.

The term “host cell” as used in the present context refers to cells which do not naturally contain the nucleic acids according to the invention.

Host cells which are suitable are prokaryotic cells, preferably E. coli, and eukaryotic cells, such as cells of Saccharomyces cerevisiae, Pichia pastoris, insects, plants, frog oocytes and mammalian cell lines.

The present invention furthermore relates to polypeptides with the biological activity of a guanylate kinase which are encoded by the nucleic acids according to the invention.

Preferably, the polypeptides according to the invention comprise an amino acid sequence from phytopathogenic fungi selected from
(a) the sequence as shown in SEQ ID No: 2,
(b) sequences which have at least 80%, preferably at least 85%, especially preferably 90% and particularly preferably 95% identity with the sequence defined under a),
(c) the sequences specified under b), which comprise the motif T-[ST]-R-x(2)-[KR]-x(2)-[DE]-x(2)-G-x(2)-Y-x-[FY]-[LIVMK] or the motif T-T-R-x(2)-R-x(2)-E-x(2)-G-x(2)-Y-x-Y-V, and
(d) fragments of the sequences stated under a) to c) which have the same biological activity as the sequence defined under a).

The term “polypeptides” as used in the present context refers not only to short amino acid chains which are generally referred to as peptides, oligopeptides or oligomers, but also to longer amino acid chains which are normally referred to as proteins. It comprises amino acid chains which can be modified either by natural processes, such as post-translational processing, or by chemical prior-art methods. Such modifications may occur at various sites and repeatedly in a polypeptide, such as, for example, on the peptide backbone, on the amino acid side chain, on the amino and/or the carboxyl terminus. For example, they comprise acetylations, acylations, ADP ribosylations, amidations, covalent linkages to flavins, haem moieties, nucleotides or nucleotide derivatives, lipids or lipid derivatives or phophatidylinositol, cyclizations, disulphide bridge formations, demethylations, cystine formations, formylations, gamma-carboxylations, glycosylations, hydroxylations, iodinations, methylations, myristoylations, oxidations, proteolytic processings, phosphorylations, selenoylations and tRNA-mediated amino acid additions.

The polypeptides according to the invention may exist in the form of “mature” proteins or as parts of larger proteins, for example as fusion proteins. They can furthermore exhibit secretion or leader sequences, pro-sequences, sequences which allow simple purification, such as polyhistidine residues, or additional stabilizing amino acids. The proteins according to the invention may also exist in the form in which they are naturally present in the source organism, from which they can be obtained directly, for example. Likewise, active fragments of a guanylate kinase may be employed in the methods according to the invention, as long as they make possible the determination of the enzyme activity of the polypeptide, or its inhibition by a candidate compound.

In comparison with the corresponding regions of naturally occurring guanylate kinases, the polypeptides used in the methods according to the invention can have deletions or amino acid substitutions, as long as they still exhibit at least the biological activity of a complete guanylate kinase. Conservative substitutions are preferred. Such conservative substitutions comprise variations, one amino acid being replaced by another amino acid from the following group:
1. Small, aliphatic residues, which are non-polar or of little polarity: Ala, Ser, Thr, Pro and Gly;
2. Polar, negatively charged residues and their amides: Asp, Asn, Glu and Gln;
3. Polar, positively charged residues: His, Arg and Lys;
4. Large, aliphatic, non-polar residues: Met, Leu, Ile, Val and Cys; and
5. Aromatic residues: Phe, Tyr and Trp.

One possible guanylate kinase purification method is based on preparative electrophoresis, FPLC, HPLC (for example using gel filtration columns, reversed-phase columns or mildly hydrophobic columns), gel filtration, differential precipitation, ion-exchange chromatography or affinity chromatography (cf. Example 2).

A rapid method of isolating the guanylate kinases which are synthesized by host cells starts with expressing a fusion protein, where the fusion moiety may be purified in a simple manner by affinity purification. For example, the fusion moiety may be a MBT tag (cf. Example 2), in which case the fusion protein can be purified on amylose resin. The fusion moiety can be removed by partial proteolytic cleavage, for example at linkers between the fusion moiety and the polypeptide according to the invention which is to be purified. The linker can be designed in such a way that it includes target amino acids, such as arginine and lysine residues, which define sites for trypsin cleavage. Standard cloning methods using oligonucleotides may be employed for generating such linkers.

Other purification methods which are possible are based, in turn, on preparative electrophoresis, FPLC, HPLC (e.g. using gel filtration columns, reversed-phase columns or mildly hydrophobic columns), gel filtration, differential precipitation, ion-exchange chromatography and affinity chromatography.

The terms “isolation or purification” as used in the present context mean that the polypeptides according to the invention are separated from other proteins or other macromolecules of the cell or of the tissue. The protein content of a composition containing the polypeptides according to the invention is preferably at least 10 times, particularly preferably at least 100 times, higher than in a host cell preparation.

The polypeptides according to the invention may also be affinity-purified without fusion moieties with the aid of antibodies which bind to the polypeptides.

The method for preparing polypeptides with guanylate kinase activity, such as, for example, the polypeptide GUK1, is thus characterized in that
(a) a host cell containing at least one expressible nucleic acid sequence encoding a polypeptide from fungi with the biological activity of a guanylate kinase is cultured under conditions which ensure the expression of this nucleic acid, or
(b) an expressible nucleic acid sequence encoding a polypeptide from fungi with the biological activity of a guanylate kinase is expressed in an in-vitro system, and
(c) the polypeptide is recovered from the cell, the culture medium or the in-vitro system.

The cells thus obtained which contain the polypeptide according to the invention, or the purified polypeptide thus obtained, are suitable for use in methods for identifying guanylate kinase modulators or inhibitors.

The present invention thus also relates to the use of polypeptides from fungi, which exert at least one biological activity of a guanylate kinase in methods for identifying inhibitors of fungicides, it being possible to use the guanylate kinase inhibitors as fungicides. The Ustilago maydis guanylate kinase is especially preferably used.

Fungicidal active compounds which are found with the aid of a guanylate kinase from a specific fungal species can thus also interact with guanylate kinases from other fungal species, but the interaction with the different guanylate kinases which are present in these fungi need not always be equally pronounced. This explains inter alia the selectivity of active substances. The use, in other fungi of the active compounds which have been found with a specific guanylate kinase as a fungicide, can be attributed to the fact that guanylate kinases from different fungal species are very closely related and show pronounced homology over substantial regions. Thus, it is clear from FIG. 1 that such a homology over substantial sequence segments exists between S. cerevisiae, N. crassa, S. pombe and U. maydis and that, therefore, the effect of the substances found with the aid of, for example, U. maydis guanylate kinase is not limited to U. maydis.

The present invention therefore also relates to a method for identifying fungicides by assaying potential inhibitors or modulators of the enzyme activity of guanylate kinase (candidate compound) in a guanylate kinase inhibition test.

Methods which are suitable for identifying modulators, in particular inhibitors or antagonists, of the polypeptides according to the invention are generally based on the determination of the activity or the biological functionality of the polypeptide. Suitable for this purpose are, in principle, methods based on intact cells (in-vivo methods), but also methods which are based on the use of the polypeptide isolated from the cells, which may be present in purified or partially purified form or else as a crude extract. These cell-free in-vitro methods, like in-vivo methods, can be used on a laboratory scale, but preferably also in HTS or UHTS methods. Following the in-vivo or in-vitro-identification of modulators of the polypeptide, fungal cultures can be assayed in order to test the fungicidal activity of the compounds which have been found.

A large number of assay systems for the purpose of assaying compounds and natural extracts are preferably designed for high throughput numbers in order to maximize the number of substances assayed within a given period. Assay systems based on cell-free processes require purified or semipurified protein. They are suitable for an “initial” assay, which aims mainly at detecting a possible effect of a substance on the target protein. Once such an initial assay has taken place, and one or more compounds, extracts and the like have been found, the effect of such compounds can be studied in the laboratory in a more detailed fashion. Thus, inhibition or activation of the polypeptide according to the invention in vitro can be assayed again as a first step in order to subsequently assay the activity of the compound on the target organism, in this case one or more phytopathogenic fungi. If appropriate, the compound can then be used as starting point for the further search and development of fungicidal compounds which are based on the original structure, but are optimized with regard to, for example, activity, toxicity or selectivity.

To find modulators, for example a synthetic reaction mix (for example in-vitro transcription products) or a cellular component such as a membrane, a compartment or any other preparation containing the polypeptides according to the invention can be incubated together with an optionally labelled substrate or ligand of the polypeptides in the presence and absence of a candidate molecule. The ability of the candidate molecule to inhibit the enzyme activity of the polypeptides according to the invention can be identified for example on the basis of reduced binding of the optionally labelled ligand or a reduced conversion of the optionally labelled substrate. Molecules which inhibit the biological activity of the polypeptides according to the invention are good antagonists or inhibitors.

Detection of the biological activity of the polypeptides according to the invention can be improved by what is known as a reporter system. In this aspect, reporter systems comprise, but are not restricted to, calorimetrically or fluorimetrically detectable substrates which are converted into a product, or a reporter gene which responds to changes in the activity or the expression of the polypeptides according to the invention, or other known binding assays.

A further example of a method by which modulators of the polypeptides according to the invention can be found is a displacement assay in which the polypeptides according to the invention and a potential modulator are combined, under suitable conditions, with a molecule which is known to bind to the polypeptides according to the invention, such as a natural substrate or ligand or a substrate or ligand mimetic. The polypeptides according to the invention can themselves be labelled, for example fluorimetrically or colorimetrically, so that the number of the polypeptides which are bound to a ligand or which have undergone a conversion can be determined accurately. However, binding can likewise be monitored by means of the optionally labelled substrate, ligand or substrate analogue. The efficacy of the antagonist can be determined in this manner.

Effects such as cell toxicity are, as a rule, ignored in these in-vitro systems. The assay systems check not only inhibitory, or suppressive effects of the substances, but also stimulatory effects. The efficacy of a substance can be checked by concentration-dependent assay series. Control mixtures without test substances can be used for assessing the effects.

Owing to the host cells containing nucleic acids encoding guanylate kinase according to the invention and available with reference to the present invention, the development of cell-based assay systems for identifying substances which modulate the activity of the polypeptides according to the invention, is made possible.

The modulators to be identified are preferably small organochemical compounds.

A method for identifying a compound which modulates the activity of a fungal guanylate kinase and which can be used in crop protection as fungicide preferably consists in
a) bringing a polypeptide according to the invention or a host cell containing this polypeptide into contact with a chemical compound or a mixture of chemical compounds under conditions which permit the interaction of a chemical compound with the polypeptide,
b) comparing the activity of the polypeptide according to the invention in the absence of a chemical compound with the activity of the polypeptide according to the invention in the presence of a chemical compound or a mixture of chemical compounds, and
c) identifying the chemical compound which specifically modulates the activity of the polypeptide according to the invention, and, if appropriate,
d) subjecting the fungicidal activity of the compound identified to in-vivo tests.

In this context, the compound which specifically inhibits the activity of the polypeptide according to the invention is particularly preferably determined. The term “activity” as used in the present context refers to the biological activity of the polypeptide according to the invention.

A preferred embodiment exploits the fact that one adenosine diphosphate (ADP) molecule is liberated in the guanylate kinase reaction. The activity, or the decrease or increase in activity, of the polypeptide according to the invention can thus be determined by detecting the ADP which is being formed. Here, the lower, or inhibited, activity of the polypeptide according to the invention is monitored with the aid of the detection of the ADP being formed, by coupling with the downstream reaction of pyruvate kinase and lactate dehydrogenase. Here, pyruvate kinase converts phosphoenolpyruvate into pyruvate, which is then utilised by lactate dehydrogenase for oxidizing NADH to give NAD. The NAD concentration, which increases owing to the coupled reaction, or the NADH concentration, which decreases owing to the coupled reaction, can be measured photospectrometrically by absorption or fluorescence measurement (absorption drops at 340 nm, fluorescence drops at 340 nm (emission at 465 nm)). Here, the lower, or inhibited, activity of the polypeptide according to the invention is monitored with the aid of the photospectrometric determination of the decrease, or increase, of the NADH converted.

The measurement can also be carried out in formats conventionally used for HTS or UHTS assays, for example in microtitre plates, into which for example a total volume of 5 to 50 μl is introduced per mixture or per well and the individual components are present in one of the above-stated final concentrations (cf. Example 3). The compound (candidate molecule) to be assayed and which potentially inhibits or activates the activity of the enzyme is introduced for example in a suitable concentration in the assay buffer, which contains ATP, GMP, PEP, NADH and the auxiliary enzymes pyruvate kinase and lactate dehydrogenase. The polypeptide according to the invention is then added in the abovementioned assay buffer, thus starting the reaction. The mixture is then incubated for example for up to 30 minutes at a suitable temperature, and for example the decrease in fluorescence is measured at absorption 340 nm; emission 465 nm.

A further measurement is carried out in a corresponding mixture, but without addition of a candidate molecule and without addition of a polypeptide according to the invention (negative control). Another measurement, in turn, is carried out in the absence of a candidate molecule, but in the presence of the polypeptide according to the invention (positive control). The negative and the positive controls thus provide the reference values for the mixtures in the presence of a candidate molecule.

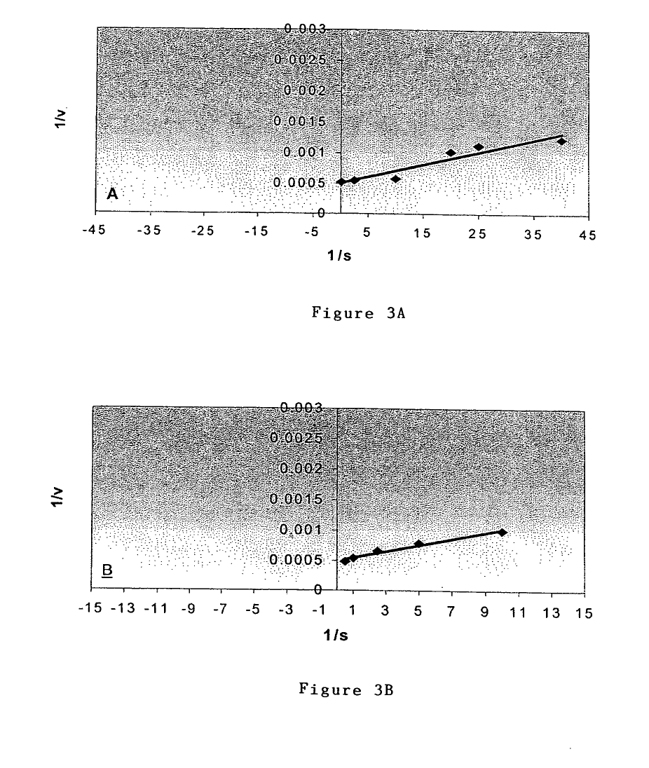

To determine optimal conditions for a method for identifying guanylate kinase inhibitors or for determining the activity of the polypeptides according to the invention, it may be advantageous to determine the K_{M} value of the polypeptide according to the invention used. This value provides information on the concentration of the substrate(s) to be used by preference. In the case of U. maydis guanylate kinase, a K_{M} of 40 μM was determined for GMP and a K_{M} of 100 μM for ATP (FIG. 3).

Compounds which inhibit fungal guanylate kinase were successfully identified within the scope of the present invention with the aid of the methods which have been described above by way of example.

In addition to the abovementioned methods for determining the enzyme activity of a guanylate kinase or the inhibition of this activity and for identifying fungicides, other methods or inhibition tests, for example methods or inhibition tests which are already known, can, of course, also be used as long as they allow the determination of the activity of a guanylate kinase and the detection of an inhibition of this activity by a candidtate compound.

It has further been found within the scope of the present invention that the inhibitors of a guanylate kinase according to the invention which have been identified with the aid of a method according to the invention are capable of damaging or destroying fungi in a suitable formulation.

To this end, a solution of the active compound to be tested may be pipetted for example into the wells of microtitre plates. After the solvent has evaporated, medium is added to each well. The medium is previously treated with a suitable concentration of spores or mycelia of the test fungus. The resulting concentrations of the active compound are, for example, 0.1, 1, 10 and 100 ppm.

The plates were subsequently incubated on a shaker at a temperature of 22° C. until sufficient growth was discernible in the untreated control.

The plates were evaluated photometrically at a wavelength of 620 nm. The dose of active compound which leads to a 50% inhibition of the fungal growth over the untreated control (ED₅₀) was calculated from the readings of the different concentrations. The present invention therefore also relates to the use of modulators of fungal guanylate kinase, preferably of guanylate kinase from phytopathogenic fungi, as fungicides. The present invention also relates to fungicides which have been identified with the aid of a method according to the invention.

Compounds which are identified with the aid of a method according to the invention and which, owing to inhibition of the fungal guanylate kinase, are fungicidally active can thus be used for the preparation of fungicidal compositions.

Depending on their respective physical and/or chemical characteristics, the active compounds which have been identified can be converted into the customary formulations, such as solutions, emulsions, suspensions, powders, foams, pastes, granules, aerosols, very fine capsules in polymeric substances and in coating compositions for seed and also ULV cold and warm fogging formulations.

These formulations are produced in a known manner, for example by mixing the active compounds with extenders, that is, liquid solvents, liquefied gases under pressure, and/or solid carriers, optionally with the use of surfactants, that is, emulsifiers and/or dispersants, and/or foam-formers. In the case of the use of water as an extender, organic solvents can, for example, also be used as cosolvents. As liquid solvents, there are suitable in the main: aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol as well as their ethers and esters, ketones, such as acetone, methyl ethyl ketone, methyl isobutyl ketone or cyclohexanone, strongly polar solvents, such as dimethylformamide and dimethyl sulphoxide, as well as water. By liquefied gaseous extenders or carriers are meant liquids which are gaseous at ambient temperature and under atmospheric pressure, for example aerosol propellants, such as halogenated hydrocarbons as well as butane, propane, nitrogen and carbon dioxide. As solid carriers there are suitable: for example ground natural minerals, such as kaolins, clays, talc, chalk, quartz, attapulgite, montmorillonite or diatomaceous earth, and ground synthetic minerals, such as highly disperse silica, alumina and silicates. As solid carriers for granules there are suitable: for example crushed and fractionated natural rocks such as calcite, marble, pumice, sepiolite and dolomite, as well as synthetic granules of inorganic and organic meals, and granules of organic material such as sawdust, coconut shells, maize cobs and tobacco stalks. As emulsifiers and/or foam-formers there are suitable: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates as well as protein hydrolysates. As dispersants there are suitable: for example lignin-sulphite waste liquors and methylcellulose.

Adhesives such as carboxymethylcellulose and natural and synthetic polymers in the form of powders, granules or latices, such as gum arabic, polyvinyl alcohol and polyvinyl acetate, as well as natural phospholipids, such as cephalins and lecithins, and synthetic phospholipids can be used in the formulations. Further additives may be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations in general contain between 0.1 and 95 per cent by weight of active compound, preferably between 0.5 and 90%.

The active compounds according to the invention, as such or in their formulations, can also be used as a mixture with known fungicides, bactericides, acaricides, nematicides or insecticides, for example in order to widen in this way the spectrum of action or to prevent the build-up of resistance. In many cases, synergistic effects are achieved, i.e. the efficacy of the mixture exceeds the efficacy of the individual components.

When employing the compounds according to the invention as fungicides, the application rates can be varied within substantial ranges, depending on the application.

All plants and plant parts may be treated in accordance with the invention. In the present context, plants are understood as meaning all plants and plant populations, such as desired and undesired wild plants or crop plants (including naturally occurring crop plants). Crop plants may be plants which can be obtained by traditional breeding and optimization methods or by biotechnological and recombinant methods or combinations of these methods, including the transgenic plants and including those plant varieties which are capable, or not capable, of protection by Plant Breeders' Rights. Plant parts are understood as meaning all aerial and subterranean parts and organs of the plants, such as shoot, leaf, flower and root, examples which are mentioned being leaves, needles, stems, stalks, flowers, fruiting bodies, fruits and seeds, and also roots, tubers and rhizomes. The plant parts also include harvested material and vegetative and generative propagation material, for example cuttings, tubers, rhizomes, slips and seeds.

The treatment according to the invention of the plants and plant parts with the active compounds is affected directly or by acting on their environment, habitat or store by the customary treatment methods, for example by dipping, spraying, vaporizing, fogging, scattering, brushing on and, in the case of propagation material, in particular seeds, furthermore by coating with one or more coats.

The examples which follow illustrate various aspects of the present invention and are not to be construed as limiting.

### EXAMPLES

### Example 1

Production of Guk 1 Knock-out Mutants in U. Maydis

Cultivation of U. Maydis

The strains were grown at 28° C. on PD-, YEPS- or suitable minimal media (Holliday, 1974; Tsukada et al., 1988). The development of dikaryotic filaments was observed after strains were placed dropwise on PD plate media containing 1% charcoal (Holliday, 1974). Pathogenicity tests were carried out as described (Gillessen et al., 1992). Overnight cultures of the strains were resuspended in a concentration of 4×10⁷ cells and injected into, or placed dropwise on, young maize plants (Gaspe Flint). At least 25 plants were infected for each strain, and developing tumours were examined after 14-21 days.

Preparation of the Knock-out Cassette

Molecular-biological standard methods were carried out as described by Sambrook et al., 1989. To produce guk 1 zero mutants, the 5′ flank and the 3′ flank of the guk 1 gene were amplified by means of PCR. The template used was genomic DNA of the strain UM518. The primers LB2, with the sequence (5′-cacggcctgagtggcccgtttgtcgcttggaatcggaggc-3′), and p15, with the sequence (5′-gcgcttctgcctcacctgtgccc-3′), were employed for the 5′ flank (1347bp). The primers RB1 (5′-gtgggccatctaggcccctcgttcactagccgacaacgcc-3′) and p13 (5′-ccaacgtagaggccgcagaagacc-3′) were used for the 3′ flank (2723bp). The cleavage sites Sfi I (a) and Sfi I (b) were introduced with the primers LB2 and RB 1. The amplicons were restricted with Sfi I and ligated with the 1884 bp Sfi I fragment from the vector pBS-isoliert (Hygromycin B cassette). The 4322 bp guk 1 knock-out cassette was amplified by carrying out a PCR with the primers LB 1 (5′-cggacttgaggagaccatcatagcc-3′) and RB2 (5′-ccctgcatgctgagcagacatgcc-3′) and employed in the subsequent transformation step (Kämper and Schreier, 2001).

Preparation of U. Maydis Protoplasts

50 ml of a culture in YEPS medium was grown at 28° C. until a cell density of approximately 5×10⁷/ml (OD 0.6 to 1.0) had been reached and then centrifuged for 7 minutes in 50 ml Falkon tubes at 2500 g (Hereaus, 3500 rpm). The cell pellet was resuspended in 25 ml of SCS buffer (20 mM sodium citrate pH 5.8, 1.0 M sorbitol (mix 20 mM sodium citrate/1.0 M sorbitol and 20 mM citric acid/1.0 M sorbitol and, using pH meter, bring to pH 5.8)), centrifuged for another 7 minutes at 2500 g (3500 rpm), and the pellet was resuspended in 2 ml of SCS buffer, pH 5.8, supplemented with 2.5 mg/ml Novozym 234. Protoplasts were released at room temperature and the process was monitored under the microscope every 5 minutes. Then, the protoplasts were mixed with 10 ml of SCS buffer and centrifuged at 1100 g (2300 rpm) for 10 minutes, and the supernatant was discarded. The pellet was carefully resuspended in 10 ml of SCS buffer and again centrifuged. The wash step with the SCS buffer was repeated twice, and the pellet was washed in 10 ml of STC buffer. Finally, the pellet was resuspended in 500 μl of cold STC buffer (10 mM Tris/HCl pH 7.5, 1.0 M sorbitol, 100 mM CaCl₂) and maintained on ice. Aliquots can be stored for several months at −80° C.

Transformation of U. Maydis

The diploid U. maydis strain was transformed by the method of Schulz et al., 1990. Genomic U. maydis DNA was isolated as described by Hoffmann and Winston 1987 or as described in the protocol of Qiagen (DNeasy-Kit).

For the transformation, not more than 10 μl of DNA (ideally 3-5 μg) were transferred into a 2 ml Eppendorf tube, 1 μl of heparin (15 μg/μl) (SIGMA H3125) was added, 50 μl of protoplasts were then added, and the mixture was incubated on ice for 10 minutes. 500 μl of 40% (w/w) PEG3350 (SIGMA P3640) in STC (filter-sterilized) were added, mixed carefully with the protoplast suspension and incubated on ice for 15 minutes. The mixture was plated onto gradient plates (bottom agar: 10 ml YEPS—1.5% agar—1M sorbitol supplemented withn antibiotic; shortly before plating, the bottom agar layer was covered with 10 ml of YEPS—1.5% agar—1 M sorbitol; the protoplasts were plated and the plates were incubated for 3-4 days at 28° C.

The homologous recombination in a genomic locus of gukl was demonstrated by means of standard methods (PCR or Southern analysis) using isolated genomic DNA. Here, it was shown that the integration of the guk1 knock-out cassette into a genomic guk1 locus has taken place and that a wild-type copy of the guk1 gene had thus been replaced, while the second copy was retained in this diploid strain. The resulting heterozygous guk1 mutants were subsequently employed in the pathogenicity test (Gillessen et al., 1992).

U. Maydis Spore Analysis

Spores were isolated from the tumours generated in the pathogenicity test. Thereafter, the resulting sporidia were isolated, and their phenotypes and genotypes were studied. The phenotype analysis was carried out by means of growth experiments on suitable complete media or minimal media (Holliday, 1974; Tsukada et al., 1988). It emerged that only 9 out of 75 sporidia which were analysed grew under selective conditions. 7 strains out of these were diploids. This was a first pointer that the phenotype of the guk1 zero mutant was lethal. The genotype studies were based on Southern analysis or PCR-based analysis of the sporidia. Here, it was found that no viable haploid strain in which only the guk1 gene had been replaced by the guk1 knock-out cassette was identified among 75 sporidia which were analysed. In those cases where the guk1 knock-out cassette had integrated homologously into the genomic locus of guk1, an additional, ectopic copy of the gene was found. Based on these results, it was concluded that the knock-out of the guk1 gene in Ustilago maydis results in a lethal phenotype.

### Example 2

Cloning, Expression and Purification of Guk1 or GUK1 from Ustilago Maydis

To clone, and express, guk1, the ORF from Ustilago maydis total RNA was amplified by RT-PCR using gene-specific primers. The corresponding DNA, a 609 bp amplicon, was cloned into the vector pDESTMC2 (Gateway Vector from Invitrogen, provided with an N-terminal MBP tag (=Maltose Binding Protein tag)). The resulting plasmid pGuk1 contains the complete coding sequence of guk1 in an N-terminal fusion with the MBP tag, which is part of the vector. The GUK1 fusion protein has a calculated mass of 64 kDa.

For the heterologous expression, the plasmid pGukl was transformed into E. coli BL21 (DE3) pLysS cells in such a way that the transformation mixture acted directly as preculture in 50 ml of selection medium. These cells were incubated overnight at 37° C. and subsequently diluted 1:25 in selection medium (LB medium supplemented with 100 μg/ml ampicillin). Induction was effected at an OD_{600 nm} of 0.8-1.0, using 1 mM IPTG (final concentration) at 28° C. After 4 hours of induction, the cells were harvested and stored at −20° C. For the disruption, the pellet was disrupted in 15 ml of lysis buffer (20 mM Tris/HCl pH 7.5; 200 mM NaCl; 1 mM EDTA +600 μl lysozyme (50 mg/ml)+150 μl DNAseI+330 μl MgCl₂ (1M)+1 mM DTT) and subsequently sonified. The cytoplasm fraction obtained by centrifugation (15 minutes at 4° C., 10 000 g) was used for isolating the expressed protein. Purification was in accordance with the standard protocol of the manufacturer for amylose resin (New England Biolabs). The purified protein was treated in buffer with glycerol (lysis buffer +10 mM maltose, 10% glycerol) and stored at −80° C. Example 3

Identification of Guanylate Kinase Modulators in 384-well MTPs in a Coupled Assay

384-well microtiter plates from Greiner were used for identifying guanylate kinase modulators.

The negative control was pipetted into the first row. The negative control was composed of 25 μl of assay buffer (100 mM Tris/HCl pH 7.7, 15 mM MgCl₂, 100 mM KCl) and 20 μl of substrate solution (250 mM Tris/HCl, pH 7.7; 250 mM KCl; 37.5 mM MgCl₂; 1.05 mM PEP; 760 μM NADH; 850 μM GMP; 2 mM ATP; 5 U/ml pyruvate kinase; 5 U/ml lactate dehydrogenase). The positive control was pipetted into the second row. The positive control was composed of 5 μl of assay buffer with 5% DMSO, and 20 μl of substrate solution (250 mM Tris/HCl, pH 7.7; 250 mM KCl; 37.5 mM MgCl₂; 1.05 mM PEP; 760 μM NADH; 850 μM GMP; 2 mM ATP; 5 U/ml pyruvate kinase; 5 U/ml lactate dehydrogenase) and 25 μl of nzyme solution (0.3 μg/ml guanylate kinase; 20% glycerol, 0.5% BSA; 100 mM Tris/HCl pH 7.7; 100 mM KCl, 15 mM MgCl₂). A test substance in a concentration of 2 μM in DMSO was introduced into the remaining rows, the assay buffer being used for diluting the substance to a volume of 5 μl. After addition of 20 μl of substrate solution (250 mM Tris/HCl, pH 7.7; 250 mM KCl; 37.5 mM MgCl₂; 1.05 mM PEP; 760 μM NADH; 850 μM GMP; 2 mM ATP; 5 U/ml pyruvate kinase; 5 U/ml lactate dehydrogenase), 25 μl of enzyme solution (0.3 μg/ml guanylate kinase; 10% glycerol, 0.5% BSA; 100 mM Tris/HCl pH 7.7; 100 mM KCl, 15 mM MgCl₂) were added to initiate the reaction.

This was followed by incubation at room temperature for 30 minutes. The NADH which was converted during the reaction was subsequently measured by determining the drop in fluorescence (absorption 340 nm; emission 465 nm) in a Tecan SPECTRAFluor Plus which is suitable for MTP.

### Example 4

Demonstration of the Fungicidal Effect of the Guanylate Kinase Inhibitors Identified

A methanolic solution of the active compound identified with the aid of a method according to the invention (Ex. 3), treated with an emulsifier, was pipetted into the wells of microtiter plates. After the solvent has evaporated, 200 μl of potatoe dextrose medium are added to each well. Suitable concentrations of spores or mycelia of the test fungus are previously added to the medium.

The resulting active compound concentrations are 0.1, 1, 10 and 100 ppm. The resulting emulsifier concentration is 300 ppm.

The plates are subsequently incubated on a shaker at a temperature of 22° C. until sufficient growth is observed in the untreated control. Evaluation is carried out photometrically at a wavelength of 620 nm. The dose of active compound which leads to 50% inhibition of the fungal growth over the untreated control (ED₅₀) is calculated from the readings of the different concentrations.

References

Berger, Albin; Schiltz, Emile; Schulz, Georg E. Guanylate kinase from Saccharomyces cerevisiae. Isolation and characterization, crystallization and preliminary x-ray analysis, amino acid sequence and comparison with adenylate kinases. European Journal of Biochemistry (1989), 184(2), 433-43.

Blaszczyk, Jaroslaw; Li, Yue; Yan, Honggao; Ji, Xinhua. Crystal Structure of Unligated Guanylate Kinase from Yeast Reveals GMP-induced Conformational Changes. Journal of Molecular Biology (2001), 307(1), 247-257.

Gillissen, B., Bergemann, J., Sandmann, C. Schroeer, M., Bölker, M., and Kahmann, R. (1992), A two-component regulatory system for self/nonself recognition in Ustilcigo maydis. Cell, 68: 647-657.

Hoffman, Charles S.; Winston, Fred. A ten-minute DNA preparation from yeast efficiently releases autonomous plasmids for transformation of Escherichia coli. Gene (1987), 57(2-3), 267-72.

Hofmann K., Bucher P., Falquet L., Bairoch A. (1999) The PROSITE database, its status in 1999. Nucleic Acids Res. 27, 215.

Holliday, R. (1974), Ustilago maydis. In King, R. C. (ed), Handbook of Genetics. Plenum, N.Y., pp. 575-595.

Kämper and Schreier (2001), Verfahren zur Herstellung von Deletionsmutanten [Method for the Generation of Deletion Mutants]; German Patent Application No. 10 133 926.3.

Konrad, Manfred. Cloning and expression of the essential gene for guanylate kinase from yeast. Journal of Biological Chemistry (1992), 267(36), 25652-5.

Li, Yue; Yanling, Zhang; Yan, Honggao. Kinetic and thermodynamic characterizations of yeast guanylate kinase. Journal of Biological Chemistry (1996), 271(45), 28038-28044.

Moriguchi, Mitsuaki; Kohno, Hirao; Kamei, Masaharu; Tochikura, Tatsurokuro. Purification and properties of guanylate kinase from bakers' yeast. Biochimica et Biophysica Acta (1981), 662(1), 165-7.

Mumberg, D., Müller, R., Funk, M.,.1995. Yeast vectors for the controlled expression of heterologous proteins in different genetic backgrounds. Gene 156, 119-122.

Sambrook, J., Fritsch, E., F. and Maniatis, T. (1989), Molecular cloning: A laboratory manual. Cold spring harbor laboratory press, Cold spring harbor, N.Y.

Schulz, B., Banuett, F., Dahl, M., Schlesinger, R., Schäfer, W., Martin, T., Herskowitz, I. and Kahmann, R. (1990), The b alleles of Ustilago maydis, whose combinations program pathogenic development, code for polypeptides containing a homeodomain-related motif. Cell 60, 295-306.

Stehle, Thilo; Schulz, Georg E. Three-dimensional structure of the complex of guanylate kinase from yeast with its substrate GMP. Journal of Molecular Biology (1990), 211(1), 249-54.

Tsukuda, T. et al. (1988) Isolation and characterization of an autonomously replicating sequence from Ustilago maydis. Mol. Cell. Biol. 8: 3703-3709.

1. A method for identifying fungicides, comprising:
(a) contacting a fungal polypeptide having the enzymatic activity of a guanylate kinase with a chemical compound or a mixture of chemical compounds under conditions which permit the interaction of the chemical compound with the polypeptide, wherein said enzymatic activity of said guanylate kinase is defined as the ability of said polypeptide in a guanylate kinase reaction to catalyze the conversion of ATP and GMP to ADP and GDP respectively;
(b) comparing the enzymatic activity of the guanylate kinase in a first guanylate kinase reaction, wherein said first guanylate kinase reaction occurs in the absence of a chemical compound with the enzymatic activity of the guanylate kinase in a second guanylate kinase reaction, wherein said second guanylate kinase reaction occurs in the presence of the chemical compound or mixture of chemical compounds; and
(c) selecting one or more chemical compounds which specifically modulate, optionally preferably inhibiting, the enzymatic activity of the guanylate kinase during said second guanylate kinase reaction.

2. The method according to claim 1, wherein said modulation of said enzymatic activity of the guanylate kinase is determined by:
(a) converting said ADP which is formed in the guanylate kinase reaction into said ATP with the aid of a pyruvate kinase to form a resulting pyruvate,
(b) converting the resulting pyruvate into a lactate with the aid of a lactate dehydrogenase with a corresponding consumption of NADH during said conversion, and
(c) monitoring the consumption of said NADH, optionally photospectrometrically by absorption or fluorescence measurement,
whereupon as enzymatic activity is inhibited by said one or more chemical compounds, less ADP is formed and less NADH is consumed, resulting in a higher concentration of NADH with increased inhibition.

3. The method according to claim 2, wherein an inhibition of the enzymatic activity is determined from a lower increase in the ADP concentration.

4. The method according to any one of claims 1 to 3, further comprising the step of testing the fungicidal activity of the chemical compound selected by bringing it into contact with a fungus.

5. The method according to claim 4 wherein the guanylate kinase is a guanylate kinase from a phytopathogenic fungus.

6. A fungicide identified by the method according to claim 1.

7. A fungicide identified by the method according to claim 4.

8. A method for combating plant pathogenic fungi comprising applying to said fungi, a planted infected with said fungi and/or an environment of said fungi and/or said plant an inhibitor of fungal guanylate kinase.

9. The method of claim 8 wherein said inhibitor is identified by the method according to claim 1.

10. A fungicidal composition comprising a fungicide identified by the method of claim 1 and one or more extenders and/or surfactants.

11. A nucleic acid encoding a polypeptide with the biological activity of a guanylate kinase, wherein said nucleic acid is derived from a phytopathogenic fungus.

12. The nucleic acid according to claim 11, comprising a sequence selected from:
a) a sequence as shown in SEQ ID No: 1,
b) sequences encoding a polypeptide which comprises the amino acid sequence shown in SEQ ID No: 2,
c) sequences encoding a polypeptide which comprises the motif T-[ST]-R-x(2)-[KR]-x(2)-[DE]-x(2)-G-x(2)-Y-x-[FY]-[LIVMK] or the motif T-T-R-x(2)-R-x(2)-E-x(2)-G-x(2)-Y-x-Y-V,
d) sequences which hybridize with the sequences defined under a) and b) at a hybridization temperature of 42-65° C., and
e) sequences which have at least 80%, preferably at least 85% and especially preferably at least 90% identity with the sequences defined under a) and b).

13. A DNA construct comprising a nucleic acid according to any one of claim 11 or 12 and a heterologous promoter.

14. A vector comprising a nucleic acid according to any one of claim 11 or 12, or a DNA construct according to claim 13.

15. A vector according to claim 14, wherein said nucleic acid is linked functionally with regulatory sequences which ensure the expression of the nucleic acid in prokaryotic or eukaryotic cells.

16. A host cell containing a nucleic acid according to any one of claim 11 or 12, a DNA construct according to claim 13 or a vector according to any one of claim 14 or 15.

17. A polypeptide having the biological activity of a guanylate kinase, which polypeptide is encoded by a nucleic acid according to any one of claim 11 or 12.

18. A polypeptide having the biological activity of a guanylate kinase, which polypeptide comprises an amino acid sequence as shown in SEQ ID No: 2.
